(19) United States
(12) Patent Application Publication
(10) Pub. No.: US 2006/0020238 A1
(43) Pub. Date: Jan. 26, 2006

(54) Applicator with interchangeable inserts

(76) Inventors: Garry Tsaur, Rowland Heights, CA (US); Shun-I Pan, San Gabriel, CA (US); Tawei Tsao, Diamond Bar, CA (US)

Correspondence Address: JOE NIEH, 18760 E. AMAR ROAD #204, WALNUT, CA, 91789, US

(21) Appl. No.: 10/897,226

(22) Filed: Jul. 21, 2004

(51) Int. Cl. A61M 35/00

(52) U.S. Cl. 604/2

(57) ABSTRACT

The applicator with interchangeable inserts comprises of an applicator and one or more interchangeable inserts. The applicator comprises of an elongated hollow tube with an absorbent applicator tip affixed to one end. The interchangeable inserts, each comprises of an elongated tubular housing with a sealed end and an open end wherein a fluid is enclosed near the sealed end with a viscous substance disposed near the open end sealing the fluid within the elongated tubular housing. The elongated tubular housing is further provided with an opening means such as a score line near the sealed end to allow release of the enclosed fluid through the open end of the elongated tubular housing.

### BACKGROUND

1. Field of Invention

The present invention relates generally to an applicator with interchangeable containers that may be inserted into the applicator. More specifically, the applicator with interchangeable inserts relates to an absorbent swab applicator with a hollow interior to accept insertion of an interchangeable container.

2. Description of Related Art

Swab applicators such as the cotton swab applicators are commonly used to clean small surfaces and apply chemicals such as medications to wounds and other small surfaces. The most common type of cotton swab applicator comprises of a solid elongated round stick with a ball of cotton affixed to each of its ends. The end of the cotton swab applicator is generally dipped into a container of the chemical or medication to retrieve a small quantity of the same to be applied to a surface. The dipping of the end of the cotton swab applicator into the container contaminates and shortens the shelf life of the remaining contents of the container. Furthermore, it is inconvenient to carry the container with the remaining contents after the required amount is retrieved and applied.

The conventional use of the cotton swab applicator with two or more chemicals or medication is also virtually impossible due to contamination of the contents of one container with the chemical or medication from the other container. Two separate cotton swab applicators will have to be used with one cotton swab applicator used for each chemical or medication. Therefore, using conventional swab applicators, two cotton swab applicators are necessary along with two separate relatively large containers of the chemicals or medications to be applied. After use, the two relatively large containers with their remaining contents must be retained and transported if they are used away from their normal storage locations. This is not only inconvenient but also the risk of contamination to the remaining contents in the two containers is very high.

### BRIEF SUMMARY OF THE INVENTION

The applicator with interchangeable inserts comprises of an applicator comprising an elongated hollow tube with an absorbent applicator tip affixed to one end. The applicator with interchangeable inserts further comprises of one or more inserts, each comprising an elongated tubular housing with a sealed end and an open end wherein a fluid is enclosed near the sealed end with a viscous substance disposed near the open end sealing the fluid within the elongated tubular housing. The elongated tubular housing has a smaller outside dimension than the inside dimension of the applicator. The elongated tubular housing is further provided with an opening means such as a score line near the sealed end to allow release of the enclosed fluid through the open end of the elongated tubular housing. Multiple interchangeable inserts may be provided to be selectively inserted into the applicator. After the insert is inserted into the applicator, the opening means on the insert is operated to release the enclosed fluid into the applicator tip. Multiple interchangeable inserts may be simultaneously inserted into an applicator that has a sufficiently large inside dimension to accommodate the multiple inserts.

### BRIEF DESCRIPTION OF THE DRAWINGS

FIG. 1 shows the preferred embodiment of the applicator.

FIG. 2 shows the preferred embodiment of the interchangeable insert.

FIG. 3 shows the preferred embodiment of the applicator with an interchangeable insert inserted into the applicator.

FIG. 4 shows another embodiment of the applicator with an interchangeable insert inserted into the applicator.

FIG. 5 shows another embodiment of the applicator with two interchangeable inserts inserted into the applicator.

### DETAILED DESCRIPTION OF THE PREFERRED EMBODIMENT

The following description and figures are meant to be illustrative only and not limiting. Other embodiments of this invention will be apparent to those of ordinary skill in the art in view of this description.

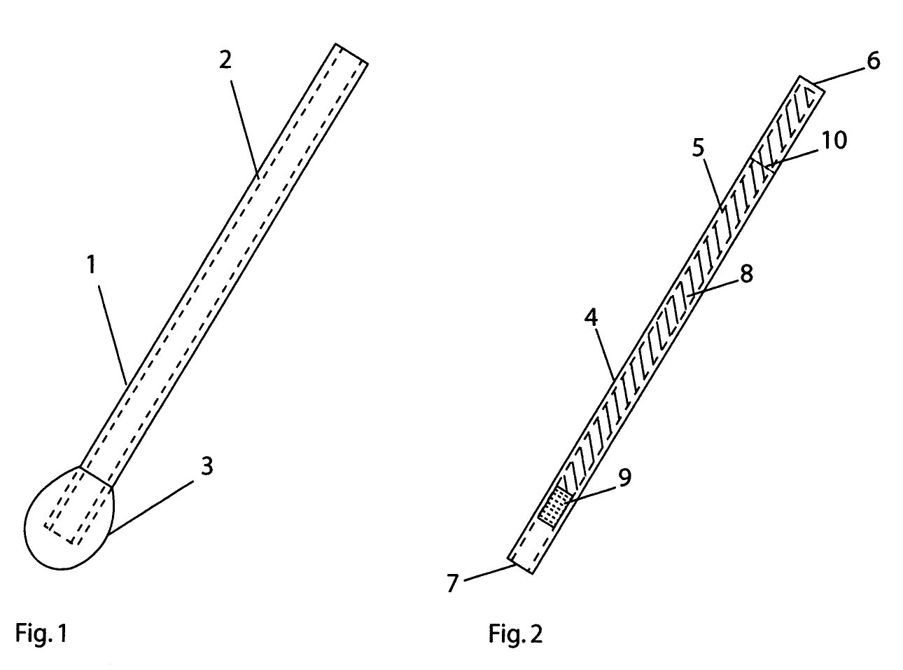

FIG. 1 shows the preferred embodiment of the applicator. The applicator with interchangeable inserts comprises of an applicator 1 comprising an elongated hollow tube 2 with an absorbent applicator tip 3 affixed to one end. Preferably the elongated hollow tube 2 is made of a flexible plastic material such as polypropylene that will bend without fracturing. Also preferably, the absorbent applicator tip 3 is made of cotton or a foam material. However, the applicator tip 3 may also be a nozzle or an opening.

The applicator with interchangeable inserts further comprises of one or more inserts 4, as shown in FIG. 2, each comprising an elongated tubular housing 5 with a sealed end 6 and an open end 7 wherein a fluid 8 is enclosed near the sealed end 6 with a viscous substance 9 disposed near the open end 7 sealing the fluid 8 within the elongated tubular housing 5. Preferably the elongated tubular housing 5 is made of a plastic material such as polypropylene. The viscous substance 9 is preferably silicone, but any other suitable viscous substance that will separate the fluid 8 in the elongated tubular housing 5 from the outside environment may be used. The elongated tubular housing 5 has a smaller outside dimension than the inside dimension of the applicator 1 such that it can slide into the applicator 1. The elongated tubular housing 5 is further provided with an opening means 10 near the sealed end 6 to allow release of the enclosed fluid 8 through the open end 7 of the elongated tubular housing 5. The preferred opening means 10 is a score line that will fracture upon bending of the elongated tubular housing 5 near the score line. However, any other suitable opening means such as a screw-on cap or a removable cap may be used.

In an alternative embodiment, the one or more inserts is an elongated tubular housing with a first sealed end and a second sealed end that is sealed with an opening mean in the form of a pressure rupturable seal enclosing a fluid. Preferably the elongated tubular housing is made of a plastic material that allows compression of the elongated tubular housing. The pressure rupturable seal may be opened by compressing the elongated tubular housing thereby increasing the fluid pressure exerted against the pressure rupturable seal thereby rupturing the pressure rupturable seal.

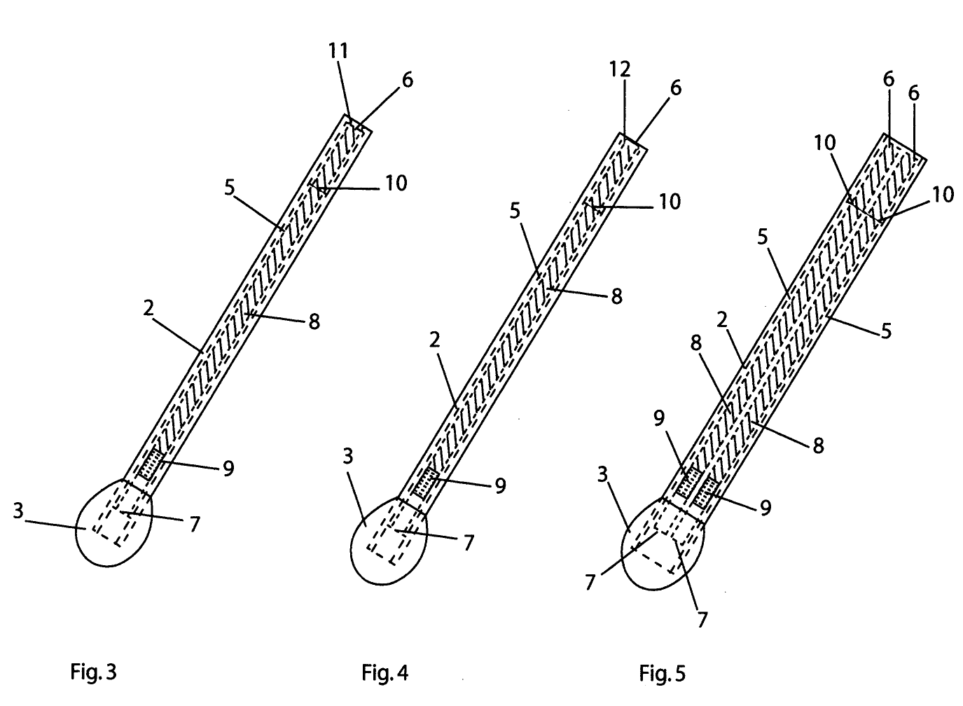

In operating the preferred embodiment, the insert 4 is inserted into the applicator 1 with its open end 7 towards the applicator tip 3 as shown in FIGS. 3 and 4 and then the opening means 10 is operated to open the sealed end 6 of the insert 4 to release the fluid 8 in the insert 4 into the applicator tip 3 through the open end 7. In operating the alternative embodiment, the insert with the pressure rupturable seal is inserted with the second end with the pressure rupturable seal towards the applicator tip and then compressed to open the second sealed end by rupturing the pressure rupturable seal. As shown in FIG. 3, a restriction 11 may be formed in the applicator 1 at or near the end opposite the applicator tip 3 such that when the sealed end 6 of the insert 4 is inserted into the applicator 1 past the restriction 11, the restriction 11 will retain the insert 4 in the applicator 1. If sealing is desired, the restriction 11 may be in the form of a ring that will seal around the insert 4 and prevent back-flow of the fluid 8 out of the end of the applicator 1.

In another embodiment, the sealed end 6 of the insert 4 may have a protrusion 12 such that when it is inserted into the applicator 1, the protrusion 12 will create an interference fit between the insert 4 and the applicator 1. As shown in FIG. 4, the protrusion 12 may be in the form of a ring near the sealed end 6 of the insert 4 such that when the insert 4 is inserted into the applicator 1, the ring will seal the open end of the applicator 1.

As shown in FIG. 5, multiple interchangeable inserts 4 may be provided to be selectively inserted into the applicator 1. After the inserts 4 are inserted into the applicator 1, the opening means 10 on the inserts 4 are operated to release the enclosed fluids 8 into the applicator tip 3. Multiple interchangeable inserts 4 may be simultaneously inserted into an applicator 1 that has a sufficiently large inside dimension to accommodate the multiple inserts 4. The applicator 1 may have an elliptical cross-section to accommodate two inserts 4. The applicator 1 may have a triangular cross-section to accommodate three inserts 4. The applicator 1 may have a square cross-section to accommodate four inserts 4.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An applicator with interchangeable inserts comprising:
an applicator comprising an elongated hollow tube with an absorbent applicator tip affixed to one end; and
one or more inserts, each comprising an elongated tubular housing with a sealed end and an open end wherein a fluid is enclosed near the sealed end with a viscous substance disposed near the open end sealing the fluid within the elongated tubular housing and an opening means positioned near said sealed end;
wherein said elongated tubular housing has a smaller outside dimension than the inside dimension of the applicator.

2. An applicator with interchangeable inserts as in claim 1, wherein said applicator is made of a flexible material.

3. An applicator with interchangeable inserts as in claim 2, wherein said applicator is made of plastic.

4. An applicator with interchangeable inserts as in claim 2, wherein said applicator is made of polypropylene.

5. An applicator with interchangeable inserts as in claim 1, wherein said applicator tip is made of cotton or foam.

6. An applicator with interchangeable inserts as in claim 1, wherein said viscous substance is silicone.

7. An applicator with interchangeable inserts as in claim 1, wherein said opening means is a score line.

8. An applicator with interchangeable inserts as in claim 1, wherein said opening means is a screw-on cap or a removable cap.

9. An applicator with interchangeable inserts as in claim 1, wherein multiple inserts are provided to be inserted into the applicator.

10. An applicator with interchangeable inserts as in claim 1, wherein said applicator has an elliptical cross-section.

11. An applicator with interchangeable inserts as in claim 1, wherein said applicator has a triangular cross-section.

12. An applicator with interchangeable inserts as in claim 1, wherein said applicator has a square cross-section.

13. An applicator with interchangeable inserts as in claim 1, wherein a restriction is positioned in said applicator at or near the end opposite said applicator tip.

14. An applicator with interchangeable inserts as in claim 1, wherein said sealed end of said insert has a protrusion such that when said insert is inserted into said applicator the protrusion will create an interference fit between said insert and said applicator.

15. An applicator with interchangeable inserts comprising:
an applicator comprising an elongated hollow tube with an absorbent applicator tip affixed to one end; and
one or more inserts, each comprising an elongated tubular housing with a first sealed end and a second sealed end that is sealed with an opening mean in the form of a pressure rupturable seal wherein a fluid is enclosed within said elongated tubular housing;
wherein said elongated tubular housing has a smaller outside dimension than the inside dimension of the applicator.

16. An applicator with interchangeable inserts as in claim 15, wherein said applicator is made of a flexible material.

17. An applicator with interchangeable inserts as in claim 16, wherein said applicator is made of plastic.

18. An applicator with interchangeable inserts as in claim 16, wherein said applicator is made of polypropylene.

19. An applicator with interchangeable inserts as in claim 15, wherein said applicator tip is made of cotton or foam.

20. An applicator with interchangeable inserts as in claim 15, wherein a restriction is positioned in said applicator at or near the end opposite said applicator tip.

21. An applicator with interchangeable inserts as in claim 15, wherein said first sealed end of said insert has a protrusion such that when said insert is inserted into said applicator the protrusion will create an interference fit between said insert and said applicator.
